(19) United States
(12) Patent Application Publication
(10) Pub. No.: US 2004/0009146 A1
(43) Pub. Date: Jan. 15, 2004

(54) Anti-tumor vaccine and method

(76) Inventor: Osvaldo Podhajcer, Buenos Aires (AR)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/373,456

(22) Filed: Feb. 25, 2003

Related U.S. Application Data
(60) Provisional application No. 60/359,836, filed on Feb. 26, 2002.

(51) Int. Cl. A61K 48/00; A61K 38/19

(52) U.S. Cl. 424/85.1; 424/93.2

(57) ABSTRACT

A method for treating cancer by administering an effective amount of a combination of a first expression vector comprising a type 2 (Th2) cytokines coding sequence and a second expression vector comprising a type 1 (TH1) cytokines coding sequence, in a mammalian patient, wherein the type 2 cytokines coding sequence is hIL-10 and the type 1 cytokines coding sequence is hIL-12.

It is provided an anti-tumor cell vaccine comprising cells transfected with the first expression vector and cells transfected with the second expression vectors. Said cells could be autologous cells as tumor cells, tumor infiltrating lymphocytes, LAK cells, endothelium precursor cells, fibroblasts, keratinocites and dendritic cells, wherein the cells delivery an amount of hIL-12 and an amount of hIL-10.

Further, the method comprising administering an effective amount of an expression vector comprising a first type 2 (Th2) cytokines coding sequence and a second type 1 (TH1) cytokines coding sequence as IL-10 and IL-12 coding sequences.

### BACKGROUND OF THE INVENTION

1. Field of the Invention

The invention relates to the administration of two interleukins for treating cancer, which administration preferably comprises the simultaneous and/or combined administration of the interleukins. More particularly, the invention relates to delivery gene therapy with combination of genes encoding IL-10 and IL-12; and anti-tumor cell vaccines producing IL-10 and IL-12.

2. Description of the Prior Art

Despite of many reported cases of successful effect on cancer treatment aiming at evoking an anti-tumor immune response following vaccination, the ultimate goal, i.e., induction of an effective clinical relief, remains elusive (Parmiani G. et al., Hum. Gene Ther. 11: 1269, 2000; Mach N., et al., Curr. Opin. Immunol 12: 571, 2000 and Allison J. P., et al., Curr. Opin. Immunol 12: 569, 2000). Cytokine treatment of cancer was suggested as a significant approach to bypass the constraints tumor cells present to the immune system either by modulating the expression of cell surface antigens or by the breakdown of the immunological tolerance (Parmiani G. et al., Hum. Gene Ther. 11: 1269, 2000 and Parmiani G., et al., Gene Ther. 5: 863, 1998). An important number of pre-clinic studies have shown that cytokine administration may be potentially useful for cancer treatment (Parmiani G. et al., Hum. Gene Ther. 11: 1269, 2000; Parmiani G., et al., Gene Ther. 5: 863, 1998 and Vile R. G. et al., Gene ther. 7: 2, 2000). However, the systemic administration of cytokines in clinical settings was only marginally useful and evoked serious toxicity, hampering their use and stimulated the development of delivery mechanisms providing relatively high levels, locally (Parmiani G. et al., Hum. Gene Ther. 11: 1269, 2000; and Mach N., et al., Curr. Opin. Immunol 12: 571, 2000). Genetic vaccines are mostly based either on ex vivo gene-modified cancer cells, or professional antigen presenting cells (pAPC) expressing specific cytokines ((Parmiani G. et al., Hum. Gene Ther. 11: 1269, 2000; Mach N., et al., Curr. Opin. Immunol 12: 571, 2000, and Dranoff G. et al., Proc. Natl. Acad. Sci. USA 90: 3539, 1993). In the case of pAPC such as dendritic cells or macrophages, cytokine expression was combined with tumor antigens loading of the pAPC (Dranoff G. et al., Proc. Natl. Acad. Sci. USA 90: 3539, 1993). There are many examples in the literature showing that cytokine-gene transduced tumor cells can elicit a tumor specific immunity strong enough to prevent the outgrowth of a subsequent challenge of parental unmodified cells. However, in the vast majority of the cases, cytokine-gene modified tumor cells failed in providing an efficient antitumor response in a therapeutic setting, i.e., when the tumor is already established ((Parmiani G. et al., Hum. Gene Ther. 11: 1269, 2000; and Mach N., et al., Curr. Opin. Immunol 12: 571, 2000). Moreover, since growing cells are not allowed to be used in humans, pre-clinical studies were performed aiming at validating the therapeutic potential of inactivated (either by mytomicin C treatment or irradiation) gene-modified tumor vaccines. Under these conditions, only very few studies using GM-CSF and IFNγ have shown therapeutic benefit in different tumor models (Rossi I. et al., Clin. Exp. Metastasis 16: 123, 1998; Allione A. et al., Cancer Res. 54: 6002, 1994 and Trinchieri G. et al., Blood 84: 4008, 1994).

IL-12 is considered the most potent cytokines that play a critical role in host defense against cancer through the activation of NK and T cells as well as a humoral response (Boggio K. et al., J. Exp. Med. 188: 589, 1998).

An antiangiogenic response has been also reported although it is still unclear whether this effect is mediated by NK cells (Sgadari, C., Angiolillo, A. L., and Tosato, G. (1996). Inhibition of angiogenesis by interleukin-12 is mediated by the interferon-inducible protein 10. Blood 87:3877-3882). IL-12 might also modulate MHC expression through the stimulation of IFNγ production and to immunomodulate the cell surface expression of T cell epitopes. In certain experimental settings gene transfer of IL-12 has shown a superior therapeutic benefit compared to that induced by tumor cells producing other cytokines (Giovarelli M. et al., J. Immunol 155: 3112, 1995). We have recently shown that mice immunized against colon cancer following the initial inoculation of tumor cells producing IL-12, can develop cross protection against syngeneic non organ related tumor cells (Adris S. et al., Cancer Res. 60: 6696, 2000). Interleukin-10 has also attracted attention since in spite of the Th1-immunosuppresive and anti-inflammatory effects, ectopic expression of IL-10 in murine B16 melanoma (Gerard C M et al. Loss of tumorigenicity and increased immunogenicity induced by interleukin-10 gene transfer in B16 melanoma cells. Human Gene Ther 1996; 7: 23-31) two murine models of mammary tumors (Giovarelli M et al. J Immunol 1995; 155: 3112-3123.; Kundu N, Beaty T L, Jackson M J, Fulton A M. J Natl Cancer Inst 1996; 88: 536-541) and colon carcinoma cells (Clerici M. et al., J. Natl. Cancer Inst. 90: 261, 1998) induced the of tumorgenicity.

It is well known that interleukin 10 or interleukin 12 are employed for treating malign diseases. However, it is not disclosed in the art, namely in the bibliography, patents, etc. that the IL-10 and IL-12 may be used in combination for treating cancer and, more particularly it is not expected that this is carried out by employing a locally cytokine gene delivery with both cytokines.

Robert M. Berman et al. discloses, in The Journal of Immunology, 1996, 157: 231-238, that IL-10 and IL-12 are critically related. They saw that the co-administration of rhIL-12 and rhIL-10 in a MCA207 model (cell lines) the anti-tumor effect appear additive or synergistic. This publication, however, does not discloses the use of vectors expressing both cytokines for intratumoral or local treatments, wherein such vectors form part of a vaccine composition for local treating or cytokine gene delivery for IL-12 and IL-10 in human beings. The publication neither discloses that the combined administration of IL-12 and IL-10 induces an anti-metastatic activity.

According to the prior art, when two interleukins are simultaneously administered, with one of the interleukins evoking a Th1 immune pathway and the other one evoking a Th2 immune pathway, it is accepted that both pathways are counter-inhibited. The current paradigm assumes that the administration type 2 cytokine such as IL-10 together with the administration a type 1 cytokine such as IL-12 would produce a system wherein both cytokines will inhibit each other effect, thus resulting in the lacking of an immune response. Thus, the prior art has shown that the simultaneous administration of both interleukins would not be effective for cancer treatment.

According to the immunology paradigm two interleukins that activate simultaneously both pathways, namely Th1 and Th2, will be counter-inhibited, thus giving a null immune response.

According to the present invention the inventors show that the simultaneous and/or combined administration of IL-10, a Th2 type cytokine, and IL-12, a Th1 type cytokine, produces a synergic effect on the inhibition of growing of established tumors and metastatic capacity in animal models, remarkably reducing the tumor size or, in many cases, entirely causing the tumor to disappear from the animal.

### SUMMARY OF THE INVENTION

It is an object of the present invention to provide a method for treating cancer by administering an effective amount of a combination of a first expression vector comprising a type 2 (Th2) cytokines coding sequence, a biologically active fragment, a variant or a peptide thereof; and a second expression vector comprising a type 1 (TH1) cytokines coding sequence, a biologically active fragment, a variant or a peptide thereof, in a mammalian patient. More specifically the invention comprising a method for cancer treatment wherein the Type 2 cytokines coding sequence is hIL-10, a biologically active fragment, a variant or a peptide thereof and the Type 1 cytokines coding sequence is hIL-12, a biologically active fragment, a variant or a peptide thereof. Both vectors could be intra or peritumorally administrated alone or in a pharmaceutically acceptable carrier. The first expression vector and the second expression vector drive the expression of the encoded hIL-10 and hIL-12 cytokines, respectively. The vectors could be retrovirus, adenovirus, adenovirus associated virus, herpes virus, lentivirus, vaccinia virus or nono viaral vectors.

According to another object of the invention, the vectors could be prepared as a pharmaceutically composition comprising an effective amount of a first expression vector and a second expression vector in a pharmaceutically acceptable carrier and/or excipient.

It is another object of the invention to provide an anti-tumor cell vaccine comprising cells transfected with the first expression vector and cells transfected with the second expression vectors. Said cells could be autologous cells as tumor cells, tumor infiltrating lymphocytes, LAK cells, endothelium precursor cells, fibroblasts, keratinocites and dendritic cells, wherein the cells delivery an amount of hIL-12, a biologically active fragment, a variant or a peptide thereof and an amount of hIL-10, a biologically active fragment, a variant or a peptide thereof.

It is still another object of the invention to provide a method for treating cancer by administering close to the tumor an effective amount of the anti-tumor cell vaccine in a mammalian patient.

According to another embodiment the invention a method is provided, the method being for treating cancer and comprising administering an effective amount of an expression vector comprising a type 2 (Th2) cytokines coding sequence, a biologically active fragment, a variant or a peptide thereof; and a second type 1 (TH1) cytokines coding sequence, a biologically active fragment, a variant or a peptide thereof, in a patient in need of cancer treatment. In a preferred embodiment the Type 2 cytokine coding sequence is hIL-10, a biologically active fragment, a variant or a peptide thereof and the second Type 1 cytokine coding sequence is hIL-12, a biologically active fragment, a variant or a peptide thereof.

Also according to the invention, the expression vector could be intra or peritumorally administrated alone or in a pharmaceutically acceptable carrier. Such expression vector drives the expression of the encoded TH1 and TH2 cytokine, more particularly the vector drives the expression of the encoded hIL-10 and hIL-12 cytokines. The vector could be retrovirus, adenovirus, adenovirus associated virus, herpes virus, lentivirus, vaccinia virus or non viaral vectors. The non viral vector could be administrating into the patient by techniques as liposomes and gene gun.

According to another object of the invention, the vector could be prepared as a pharmaceutically composition comprising an effective amount of the expression vector in a pharmaceutically acceptable carrier and/or excipient.

It is also another object of the invention to provide an anti-tumor cell vaccine comprising cells transfected with an expression vector. The anti-tumor cell vaccine comprises autologous cells as tumor cells, tumor infiltrating lymphocytes, LAK cells, endothelium precursor cells, fibroblasts, keratinocites and dendritic cells, wherein the cells delivery an amount of hIL-12, a biologically active fragment, a variant or a peptide thereof and an amount of hIL-10, a biologically active fragment, a variant or a peptide thereof.

It is a further object of the invention to provide a method for treating cancer by administering, close to the tumor of a mammalian patient, an effective amount of the anti-tumor cell vaccine.

### BRIEF DESCRIPTION OF THE DRAWINGS

FIG. 1 shows the vaccination of mice with s.c. CT26 colon or LM3 mammary tumors. (A, C and E) are mice that bear CT26 tumors. (B, D and F) are mice that bear LM3 tumors. Mice with s.c. tumors after injection of 3×10⁵ cells were vaccinated once per week during four weeks with live (A and B), mitomycin c-inactivated (C and D), and γ-irradiated vaccines (E and F). p values correspond to groups showing statistically significant prolonged survival compared to their respective controls; each group contained 8 mice. Vaccines were prepared with the following cells; ε) CT26-neo or LM3-neo; ∘) CT26-IL10 or LM3-IL10; ˜) CT26-IL12 or LM3-IL12; *) CT26-(IL-10+IL12) or LM3-(IL-10+IL12). (G) The importance of local IL-10 expression on tumor rejection. Mice bearing CT26 tumors in the left flank were vaccinated with Mitomycin C—inactivated tumor vaccines as following: ▴) CT26-IL10 in the right flank; ∘) CT26-IL12 in the right flank; ˜) CT26-IL12 in the left flank and CT26-IL10 in the right flank; *) CT26-(IL-10+IL12) in the right flank; ⋄) CT26-(IL-10+IL12) in the left flank. Each group contained 10 mice. Mice were treated following institutional guidelines and hence, sacrificed when tumor size reached 2 cm³. Survival analyses were performed using Kaplan-Meyer's method. Statistical comparisons were made using the log-rank test. The figure shows one of 3 experiments with similar results.

FIG. 2 shows the evaluation of the immune infiltrate following mice treatment with the different vaccines. (A) Treatment of mice bearing CT26 tumors either with mitomycin C-inactivated CT26-IL10 or CT26-IL12 vaccines induced mainly the recruitment of neutrophils to the site of tumor growth (arrows). (B) correspond to the infiltrate observed when mice were vaccinated with the combined CT26 (IL10+IL12) MitC V vaccine; macrophages (arrow, M) stained positive with an anti-CD11b antibody, while an apparent lack of reactivity was observed in lymphocytes (arrow, L), although CD11d was described to be expressed by other immune cell types as well. (C) General view of the vaccination area showing the tumor area (arrow T), the site of injection of the combined vaccine (arrow V) and the inflammatory infiltrate close to the vaccine (arrow I). (D) Role of CD4+ and CD8+ T cells in tumor rejection following vaccination with CT26-(IL10+IL12) MitC-V vaccine. Mice were selectively depleted by in vivo treatment with specific antibodies for different lymphocytes subpopulations (Adris S. et al., Gene Ther. 6: 1705, 1999, herein incorporated by reference). Magnification (A and B) 630×; (C) 200×.

FIG. 3 shows the immune infiltrate in the lungs following mice vaccination. (A) Quantification of the neutrophil infiltrate in lungs obtained from mice vaccinated with the different types of Mit-C-inactivated vaccines. The data corresponds to the total amount of neutrophils observed in 10 different fields in 3-4 mice (mean±SD). (B) microscopic section corresponding to LM3 tumor-bearing mice vaccinated with LM3-IL12 MitC-V vaccine showing a high amount of neutrophils (see also the insert). The intraluminal areas were dramatically reduced in these groups of mice (C) microscopic section corresponding to naive mice showing no immune infiltrate and normal intraluminal areas. (D) microscopic section corresponding to LM3-tumor bearing mice vaccinated with LM3-(IL-10+IL12) MitC-C vaccine showing a vast number of lymphocytes close to an artery, surrounding few tumor cells (see also the insert; arrow L, lymphocyte; arrow T, tumor cell). The intraluminal areas were similar to those observed in naive mice. Magnification (A, B and C), 630×.

FIG. 4 shows the evaluation of T helper response as a read out of IL-10, and IL-12 activity. (A and B) Mice bearing CT26 tumors were vaccinated with MitC-V vaccines and one week after each vaccination spleen cells were obtained for cytokine quantification. (C and D) Mice bearing LM3 tumors were vaccinated with MitC-V vaccines and one week after the last vaccination spleen cells were obtained for cytokine quantification. (***) corresponds to groups showing statistically significant differences (p<0.001) compared to their respective control groups of animals vaccinated with cells expressing no cytokine. (E) total circulating and CT26-specific IgG2a and IgG1 subclass levels. Serum was obtained from each mice one week after the last vaccine.

FIG. 5 is an histogram showing the induction percentage of lymphoblasts proliferation with regard to the control of presence of supernatant cells CHO transfected with IL10-IL12, IL12 and human IL12lig.

FIG. 6 is an histogram showing the induction percentage of lymphoblasts proliferation with regard to the control of presence of supernatant cells PT67 transfected with the plasmid MVL-IL12lig.

FIG. 7 shows the cloned cDNA of IL10. Panel A shows the amplification of IL10 from cDNA of lymphocites, wherein the product of the amplification of 570 bp may be seen. Panel B shows the expected profile according to the sequence and the right side of this panel shows the restriction products obtained with the enzymes Bsu361 and CLAI. The rows indicate the positive clones for MVL-IL-10. Panel C shows plasmids pCite-1-hIL10 and the right side of this panel shows the restriction fragments obtained with enzyme XmnI, with the rows indicating fragment 530 bp.

FIG. 8 shows the cloning steps of sub-units p40 and p35 of hIL12 in plasmid pBabe-Neo. Panel A shows the amplification products corresponding to p40 and p35, respectively. Panel B shows the restriction pattern that is expected for plasmid pGEM-p35 and the right side of this panel shows the restriction fragments of pGEM-p35 with the enzyme EcoRI. Panel C shows the partial restriction profile of pCite-p40 with enzymes EcoRI and SalI, wherein the fragment that was then cloned in the plasmid pBabe-Neo is indicated. The right side of this panel shows the restriction pattern with the enzyme HindIII of a clon bearing plasmid MVL-p40. Panel D shows the isolation of sub-unit p35 from plasmid pGEM-p35 by digestion with enzymes SnaBI and BamHI and the further cloning of said fragment of 870 bp in MVL-p40. Panel E shows the restriction pattern expected for plasmid MVL-hIL12 digested with enzymes EcoRI, Hindi, BglII and XmnI, and the right side of this panel shows the results in an agarose gel of such digestion.

FIG. 9 shows the construction steps of the retroviral vector MVL-hIL12-hIL10. The cDNA of hIL10, together with sequence IRES, was amplified by PCR with primers T7/IL10R. The product of this reaction was sub-cloned in vector pGEM. Afterwards, the fragment of 1200 bp obtained from PGEM by digestion with SalI was cloned in the site SalI of plasmid MVL-hIL12.

FIG. 10 shows the steps for obtaining a unique cDNA expressing sub-units p40 and p35 of hIL-12. Panel A indicates the fragment of the primer that is homologous to the sequence, the coding region for the histidines, valines and prolines and the recognizing site XmaI. Panel B shows the different cloning steps of IL-12lig in pBabe-Neo.

FIG. 11 shows an scheme of the plasmids of the invention wherein A) is the scheme of plasmid MVL-hIL; B) is the scheme of plasmid MVL-hIL12; C) is the scheme of plasmid MVL-hIL-12-hIL10 and D) is the scheme of plasmid MVL-hIL-12lig.

### DETAILED DESCRIPTION OF THE PREFERRED EMBODIMENTS

It will be readily apparent to one skilled in the art that various substitutions and modifications may be made in the invention disclosed herein without departing from the scope and spirit of the invention.

The term “method for treating cancer” as used herein refers to method for treating a primary cancer and metastatic cancer; and a method for preventing metasteses development.

The inventors determined the therapeutic effectiveness of combining autologous tumor cell vaccines producing IL-12 and IL-10 as compared to the effectiveness of each one separately on mice bearing large established colon and mammary tumors. They also evaluated the immunological parameters associated to the antitumor response elicited by the combined vaccine compared to each single vaccine.

The inventors studied the effect of tumor-vaccine producing the combination of both cytokines in a therapeutic setting of established murine models of primary tumors and metastasis. None of the mice injected either with 3×10⁶ CT26 colon cancer (n=38) or LM3 mammary tumor (n=20) cells developed tumors, when cells were engineered to produce low amounts of IL-12. CT25-IL12 and LM3-IL12 cells produced 0.21 and 0.37 ng/ml per 10⁵ cells per 24 hr of IL-12, respectively. CT26-IL10 and LM3-IL10 produced 5.8 and 6.3 ng/ml per 105 cells per 24 hr of IL-10, respectively.

In addition, only 37% (10/19) and 54% (12/22) of mice produced slow growing tumors when injected either with 5×10⁵ CT26 or LM3 cells producing IL-10. None of mice developed tumors when injected with de combination of 5×10⁵ CT26-IL10+3×10⁶ CT26-IL12 (n=14) or 5×10⁵ LM3-IL10+3×10⁶ LM3-IL12 (n=16) cells. All mice that rejected primary tumor injection rejected a challenge with parental cells. Control mice transduced with the retroviral vector without insert (CT26-neo (n=10) and LM3-neo (n=16)), developed rapidly growing tumors.

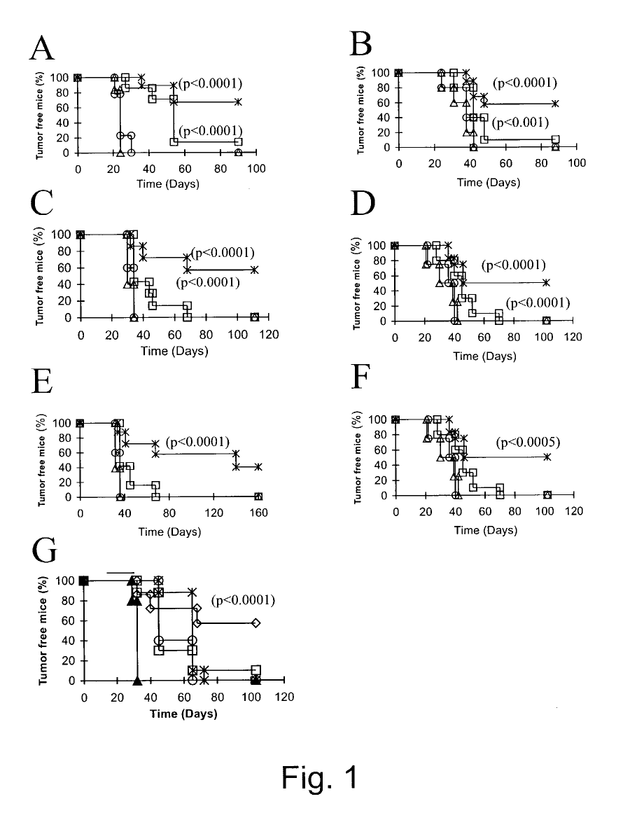

In order to establish the therapeutic efficacy of a combined vaccine producing IL-12 and IL-10, syngeneic Balb/c mice were injected s.c. with tumorigenic inocula of either CT26 or LM3 cells. After 21-22 days post-injection (average tumor size, 300-400 mm3), mice were vaccinated s.c. at a distance of 1.0-1.5 cm from the growing tumor area, with syngeneic live vaccine (LV) made either of 5×10⁵ CT26-IL10 cells, 3×10⁶ CT26-IL12 cells or their combination CT26-(IL10+IL12). None of the mice vaccinated either with control CT26-neo LV or CT26-IL10 LV benefited from vaccine administration (FIG. 1A). Mice treated with CT26-IL12 LV developed tumors with delayed growth compared to control mice and less than 10% of total mice remained free to tumor growth up to the end of experiment (FIG. 1A). All mice treated with CT26-(IL10+IL12) LV developed tumors with great delay and in 70% of them the tumor ceased growing until the end of the experiment (FIG. 1A). Similarly, vaccination of LM3-tumor bearing mice with LM3-(IL10+IL12) LV induced tumor regression in at least 60% of mice, while LM3-IL12 LV had only marginal effects and LM3-neo LV or LM3-IL10 LV had no effect at all (FIG. 1B). Ti further determine the efficacy of combining IL-10 and IL-12 gene transduced cells as a vaccine, tumor-bearing mice were vaccinated with mitomycin C-inactivated vaccines (MitC-V). In line with the therapeutic efficacy observed with LV vaccines, only CT26-(IL10+IL12)MitC-V was highly effective and cured 60% of the animals carrying established CT26 tumors (FIG. 1C). On the contrary, CT26-IL12 MitC-V vaccination was only marginally effective while CT26-neo MitC-V or CT-26-IL10 MitC-V has no effect (FIG. 1C). In coincidence, treatment of LM3-tumor bearing mice with the combined LM3-(IL10+IL12) MitC-V cured 50% of animals (FIG. 1D). On the contrary, LM3-IL12 MitC-V was slightly effective while LM3-neo MitC-V or LM3-IL10 MitC-V had essentially no effect on tumor growth (FIG. 1D).

The therapeutic efficacy of the combined vaccine was further confirmed since only a y-irradiated vaccine (γ-IV) combining both cytokines CT26-(IL10+IL12) γ-IV delayed tumor growth in 70% of mice and 40% remained free of tumor after 6 months (FIG. 1E). On the contrary, CT26-IL12 γ-IV alone was only slightly effective and CT26-neo γ-IV or CT26-IL10 γ-IV had no effect (FIG. 1E). Similarly, a LM3-(IL10+IL12) γ-IV was effective in curing 50% of mice carrying mammary tumors, while other vaccination protocols were almost ineffective (FIG. 1F). The therapeutic effectiveness of CT26-(IL10+IL12) MitC-V vaccination was strongly diminished when mice were simultaneously vaccinated with CT26-IL12 MitC-V in the ipsilateral flank and CT26-IL10 MitC-V in the opposite flank indicating a dominant role of IL-10 when expressed close to the site of tumor growth (FIG. 1G). These data demonstrated the therapeutic benefit of autologous tumor cells vaccines compared to other forms of vaccination, and confirmed that the combination of IL-10+IL-12 has superior therapeutic benefit than each single agent regardless of the type of vaccine utilized.

The inventors demonstrated that a combined vaccine producing both IL-10 and IL-12 allow a synergistic effect of the cytokines on tumor growth. Previous data from different laboratories suggested that IL 10 may be chemotactic for lymphocytes (Jinquan T et al. J Immunol 1993; 151: 4545-4551, herein incorporated as reference). In line with this, the therapeutic effectiveness of the combined CT26-(IL10+IL12) MitC-V was strongly diminished when mice were vaccinated in the opposite flank with the whole combined vaccine or when they were simultaneously vaccinated with CT26-IL12 MitC-V 1.5 cm far from the tumor area and CT26-IL10 MitC-V in the opposite flank. Moreover, only conditioned media obtained either from proliferating or Mit C-inactivated CT26-IL10 and LM3-IL10 cells stimulated the chemotactic migration of PBL (data no shown) suggesting that in vivo IL-10 may act at least in part as a lymphocyte chemo-attractant.

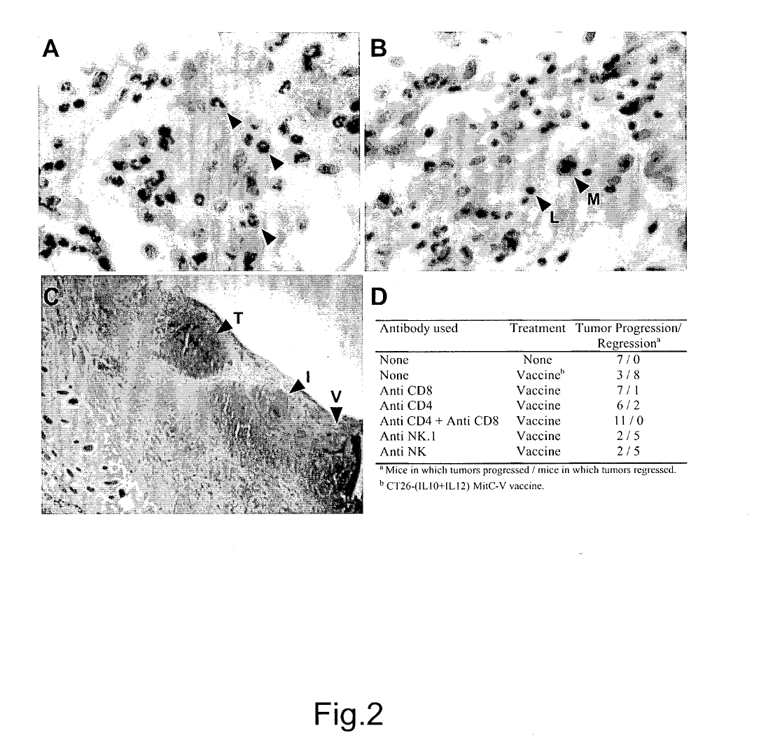

Kinetics histological studies were performed of the inflammatory response recruited to the area of tumor growth following vaccine administration. Three-fold and 10-fold increased PMN neutrophil recruitment to the periphery of the tumor area, were observed after the first vaccination with CT26-IL10 MitC-V and CT26-IL12 MitC-V, respectively (FIG. 2A), compared to control mice which showed almost no inflammatory infiltrate all along the vaccination regime. An expected decrease in PMN neutrophils infiltrate was observed after the third vaccination, with a slight increase in the amount of infiltrating lymphocytes and macrophages which was very similar in the CT26-IL10 MitC-V and the CT26-IL12 MitC-V-treated groups (data not shown). However, vaccination with the combined CT26-(IL10+IL12) MitC-V vaccine induced immediately after the first vaccine, 12-fold, 8-fold and 3-fold increased recruitment of neutrophils, macrophages and lymphocytes, respectively, compared to control mice (FIG. 2B). A further increase in the amount of recruited macrophages was observed after the second vaccine which then remained stable, while a steadily increase in lymphocytes recruitment was observed up to the third vaccine (not shown). We were unable to assess the amount of infiltrating immune cells after the fourth CT26-(IL10+IL12) MitC-V vaccine due to the large tumor necrosis. Almost 100% of the tumor area was necrotic after the fourth combined vaccine, compared to 30% or less in the other groups of mice. Administration of the combined vaccine was characterized by a dense immune infiltrate surrounding the area of the vaccine immediately after the first administration (FIG. 2C). In order to identify the subset of immune cells responsible for the antitumor effect, we depleted mice with specific antibodies. The simultaneous depletion of CD4+ and CD8+ cells by antibody injection completely abolished the therapeutic effectiveness of the CT26-(IL10+IL12) MitC-V combined vaccine (FIG. 2D).

LM3 cells develop spontaneously a small number of large lung metastases in less than 20 days after s.c. injection in the flank (Table 1).

TABLE 1
Development of spontaneous lung metastases
after s.c. injection of LM3 cells.

| | Mice with lung | |
|---|---|---|
| Days^{a} | metastasis^{b} | Nodules/mice |
| 13 | 4/5 | 1-2 |
| 19 | 6/6 | 1-3 |
| 23 | 4/4 | 2-3 |
| 27 | 4/4 | 1-5 |
| 30 | 2/2 | 3-5 |
| 39 | 3/3 | 2-6 |

^{a}Groups of mice were sacrificed at different days after s.c. injection of 5 × 10⁵ LM3 mammary tumor cells and histological analyzed for development of spontaneous lung metastases.
^{b}Mice bearing metastases/total mice.

In order to establish if the combined vaccine can be therapeutically effective on established lung metastases, mice injected s.c. with LM3 cells were vaccinated with MiC-V vaccines starting 21 to 22 days after s.c. injection of tumor cells. None of the animals vaccinated with the combined LM3-(IL10+IL12) MitC-V vaccine developed lung metastases, compared to almost 70% of those vaccinated with LM3-IL12 MitC-V vaccine and 100% of mice corresponding to the two other groups (Table 2).

TABLE 2
Effect of mitomycin C-inactivated vaccines
on the development of spontaneous lung metastases.

| Type of vaccine | Mice with lung metastasis^{a} | Nodules/mice |
|---|---|---|
| LM3-neo | 5/5 | 1-3 |
| LM3-IL10 | 6/6 | 1-6 |
| LM3-IL12 | 4/6 | 1-4 |
| LM3-IL10 + IL12 | 0/12 | 0 |

^{a}Mice bearing/total mice. Mice were s.c. injected with 5 × 10⁵ LM3 cells, vaccinated at day 22 with the different vaccines and sacrificed at day 42 when primary tumors in control mice treated with LM3-neo MitC-V vaccine reached an average size of 2 cm³.

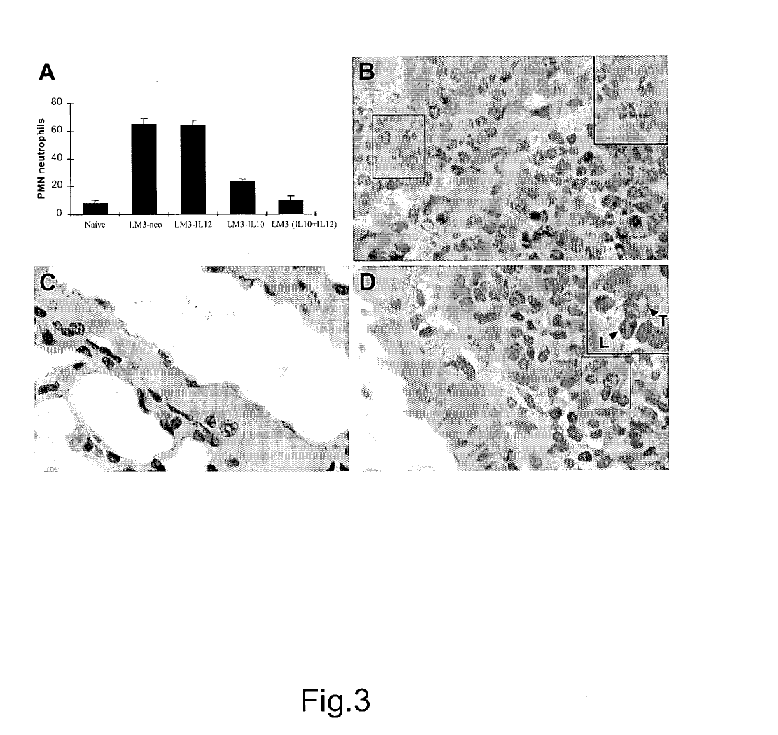

Histological analysis of the lungs showed remarkable results. Control animals vaccinated with LM3-neo MitC-V and the group vaccinated with LM3-IL12 MitC-V showed a similar level of neutrophils infiltrating the capillary network of interalveolar septa (FIG. 3, A and B). Neutrophils were not preferentially localized in contact with metastatic nodules which were mainly located in the subpleural region (data not shown). On the contrary, the groups vaccinated with LM3-IL10 MitC-V and the combined vaccine showed neutrophils infiltrate levels close to those observed in lungs from naive mice (FIG. 3, A-C). Conversely, a dramatic increase in the perivascular lymphocyte infiltrate was observed in lungs of animals vaccinated with the combined vaccine. Lymphocytes were located surrounding vessels and arteries in more than 70% of lung blood vessels of mice vaccinated with LM3- (IL10+IL12) MitC-V vaccine (FIG. 3D) compared with 7-10% in the LM3-neo, and LM3-IL12 MitC-V-treated groups, and 0% in the LM3-IL10 MitC-V-treated group. Careful histological analysis of the entire block of paraffin-embedded lungs obtained from all the mice vaccinated with LM3-(IL10+IL12) MitC-V vaccine, demonstrated intraluminal lymphocytes, and in some areas, the presence of few tumor cells surrounded by lymphocytes (FIG. 3D).

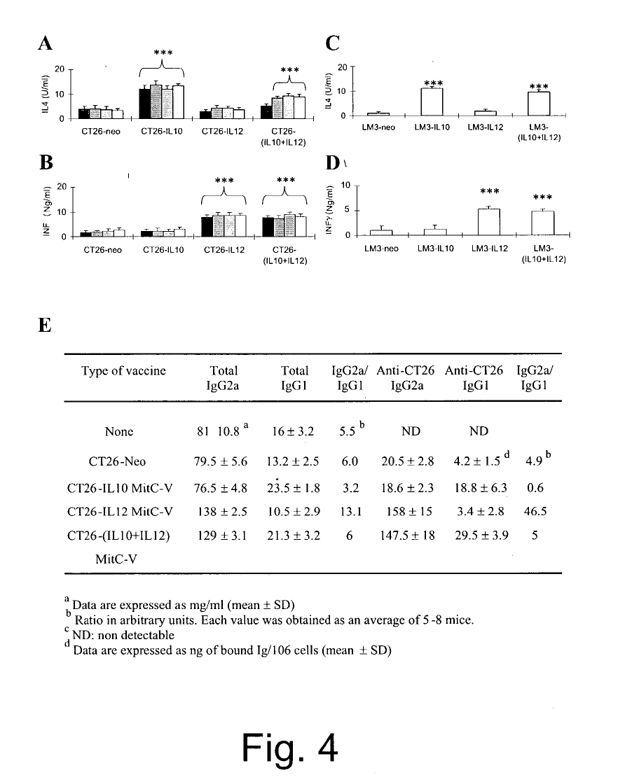

In view of the intriguing synergism observed with the combined vaccine, we assessed whether a specific T helper response prevails when these antagonistic cytokines are simultaneously produced. We observed an increased production of IL-4 by spleen cells obtained from mice vaccinated with CT26-IL10 MitC-V or the combined CT26-IL10+IL12 MitC-V vaccine, which remained stable up to the end of the experiments at days 49-50 (FIG. 4A). On the contrary, an increased IFNγ production was observed in spleen cells obtained from mice vaccinated with CT26-IL12 MitC-V or the combined vaccine (FIG. 4B). Similarly, increased production of IL-4 was observed in spleen cells only when mice received LM3-IL10 MitC-V vaccines or the combined LM3-IL10+IL12 MitC-V vaccine (FIG. 4C). Increased IFN, production was observed only after vaccination with LM3-IL12 MitC-V or the combined vaccine (FIG. 4D). Thus, the combined vaccine stimulated the overproduction of IL-4 and IFNγ by spleen cells, suggesting that both cytokines were active simultaneously. In line with this, vaccination with CT26-IL10 MitC-V induced 2-fold increase in total circulating IgG1, and 7-fold increase in CT26-specific IgG1, leading to a clear shift towards a Th2 response (FIG. 4E). Vaccination with CT26-IL12 MitC-V led to a 2-fold increase in circulating, and 8-fold increase in CT26-specific IgG2a levels, and a decrease both in circulating and CT26-specific IgG1 levels; overall, a clear shift towards a Th1 response was observed (FIG. 4E). Vaccination with the combined CT26-IL10+IL12 MitC-V led to an increase in IgG2a and IgG1 levels similar to those observed with each vaccine separately, suggesting again that both cytokines were simultaneously active (FIG. 4E).

In cancer there is a perception that a shift to a Th1 response will be of benefit to achieve an antitumor effect while a Th2 response will be detrimental. It was suggested that human neoplasia is associated with dysregulation of the theoretical equilibrium between the production of type 1 (Th1) and type 2 (Th2) cytokines (Clerici M. et al., J. Natl Cancer Inst. 90: 261, 1998, herein incorporated as reference). Production of type 2 cytokines such as IL-10 was associated with cancer progression (reviewed in Clerici M. et al., J. Natl Cancer Inst. 90: 261, 1998 herein incorporated as reference). However, qualitative impairment of balance between type1 and type2 cytokines described in cancer patients are probably the result of portraits of advanced diseases in which the tumor itself contributed to cytokine imbalance by secreting immunoregulatory factors. The present data demonstrates that this situation should not preclude to attack the tumor by using IL-10 gene expression which can synergize and strongly improve the therapeutic effectiveness of a tumor cell vaccine producing one of the most potent know type 1 cytokines, IL-12. IL-10 appears to act as a potent chemotactic adjuvant, stimulating a dramatic and immediate recruitment of immune cells which might be stimulated to attack the tumor by the expression of IL-12 in the vicinity of the tumor. While neutrophils, as other inflammatory cells, might have a role in the innate antitumor response against the primary tumor, T cells appear to play a major role in the complete remission of established primary tumors and metastases. From the tumors immunology perspective, it is remarkable that the rejection of established primary tumors and lung metastases occurred in a context in which each cytokine induced its own and specific lung lasting Th response with no evidence of counter-inhibition.

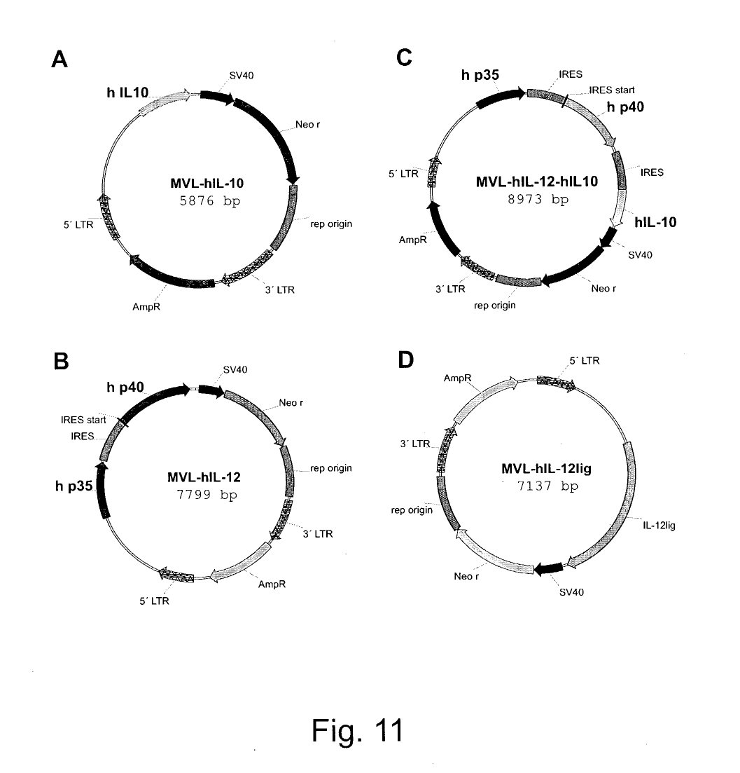

In order to prepare appropriate vector for use in gene delivery therapy in human beings, plasmids MVL-hIL12, MVL-hIL10, MVL-hIL12-hIL10 and MVL-hIL12lig have constructed as it is disclosed in Example 4 and shown in FIG. 11, these corresponding to the insertion of cDNA of the heterodimer of hIL-12 linked by a proteic bridge respectively within plasmid pBabe-Neo (Morgenstern J P, Land H. Nucleic Acid Res 1990; 18: 3587-3596, herein incorporated as reference.

For amplifying the cDNA of the cytokines hIL-10 and hIL-12, the following primers of Table 3 have been designed:

TABLE 3

Primers sequences.

| Name | Sequence 5′-3′ | |
|---|---|---|
| IL10R | GGTCGACTCGCCACCCTGATGTCTCAG | (SalI)^{a} |
| IL10F | CCCATGGGAAGGCATGCACAGCTCAGCACTGC | (NcoI) |
| P40R | GGATATCGGATCAGAACCTAACTGCAGGGC | (EcoRV) |
| P40F | CCCCATGGAGAGCAAGATGTGTCACCAGC | (NcoI) |
| P35R | CGCTACGTATTCTTAGCAATTCATTCATG | (SnaBI) |
| P35P3 | GCCGGATCCGCGGCCGCAGCATGTGTCCAGCGC | (BamHI and |
| | | Not I) |
| p40Rligant | CGCCCGGGTACCCCTACTCCAGGAACACTGCAGGGCACAGATGCCC | (XmaI) |
| P35fligant | CGCCCGGGGTTGGTGGTGCCAGAAACCTCCC | (XmaI) |
| SP6 | GATTTAGGTGACACTATAG | |
| T7 | TAATACGACTCACTATAGGG | |

The restriction enzyme for which the recognizing site has been introduced in the sequence is indicated between parenthesis and the shadow indicates the recognizing site.

Table 4 shows the experimental conditions employed in the PCR reactions for the amplifications of cDNA of hIL-10 and hIL-12.

TABLE 4
Conditions for PCR reaction.

| | Primers | | MgCl₂ | Taq | Pfu | amplifying |
|---|---|---|---|---|---|---|
| Reaction | (μM) | dNTPs (mM) | (mM) | (U) | (U) | cycle |
| IL10F/IL10R | 0, 5 c/u | 0, 2 c/u | 2 | — | 3 | 94° C., 3′; |
| | | | | | | (94° C., 1′; |
| | | | | | | 62° C., 1′; |
| | | | | | | 72° C., 2′) × 35; |
| | | | | | | 72° C., 10′ |
| P40F/P40R | 0, 5 c/u | 0, 2 c/u | 1.5 | — | 3 | 94° C., 3′; |
| | | | | | | {94° C., 1′, 59 |
| | | | | | | ° C., 1′; 72° C., |
| | | | | | | 2′} × 35; 72° C., |
| | | | | | | 10′ |
| P35F3/P35R | 0, 5 c/u | 0, 2 c/u | 2 | 2 (final | 1, 5 | 94° C., 3′; |
| | | | | 50 μl) | (final | {94° C., 1′, 60° |
| | | | | | 50 μl) | C., 1′, 72° C., |
| | | | | | | 2} × 10; {94 |
| | | | | | | ° C., 1′, 55° C., |
| | | | | | | 1′; 72° C., 2′} × |
| | | | | | | 25; 72° C., 10′ |
| P40F/p40Rlig | 0, 5 c/u | 0, 2 c/u | 1.5 | 2 (final | 1, 5 | 94° C., 3′; |
| | | | | 50 μl) | (final | {94° C., 1′, 57° |
| | | | | | 50 μl) | C., 1′; 72° C., |
| | | | | | | 2′} × 35; 72° C., |
| | | | | | | 10′ |
| P35flig/P35R | 0, 5 c/u | 0, 2 c/u | 1.5 | 2 (final | 1, 5 | 94° C., 3′; |
| | | | | 50 μl) | (final | {94° C., 1′, 55° |
| | | | | | 50 μl) | C., 1′, 72° C., |
| | | | | | | 2′} × 35; 72° C., |
| | | | | | | 10′ |
| T7/IL10R | 0, 5 c/u | 0, 2 c/u | 1.5 | 2, 5 | 2 (final | 94° C., 3′; |
| | | | | (final | 50 μl) | {94° C., 1′, 55° |
| | | | | 50 μl) | | C., 1′; 72° C., |
| | | | | | | 2′} × 35; 72° C., |
| | | | | | | 10′ |

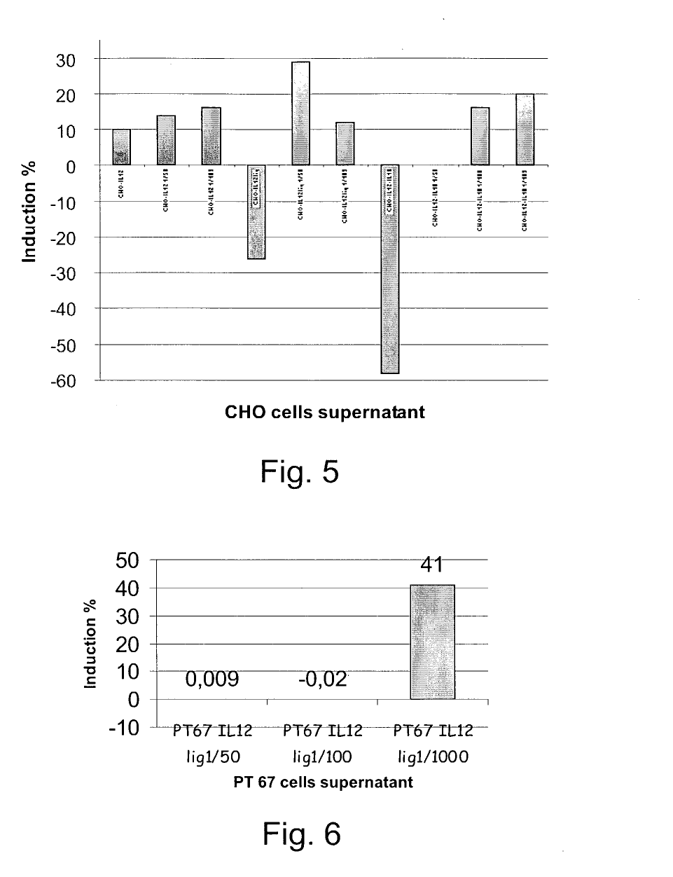

With the purpose of measuring the biological activity of cloned hIL12 in the different plasmidic constructions, two cell lines, namely CHO and PT67, were transfected with vectors MVL-hIL-12, MVL-hIL-10, MVL-hIL-12-hIL-10 y MVL-hIL-12lig. The proliferation test for PHA activated lymphocites (Example 6) was carried out by using the supernatant of the cells transfected with the different vectors. Said test demonstrates that the transfected cells supernatant yields cloned cytokines hIL-12, hIL-12lig and hIL-12 combined with hIL-10) and has biological activity (FIGS. 5 and 6).

In the CHO cells an inhibition in the growth of lymphoblasts in presence of the supernatants of cultures without diluting has been observed. As the dilution of the supernatant was diluted the inhibition was lost and an increasing in the proliferation was determined (FIG. 5). This supernatant was conserved and the IL12 concentration was measured by method ELISA, thus confirming the presence of IL12 in the samples (see ELISA results below). It has been found that the supernatants of the cells transfected with MVL-hIL-12 y MVL-hIL-12lig also induce the proliferation. In the case of MVL-hIL-12lig it has been found that the induction reaches a maximum value at a dilution that is smaller as compared to the other samples (1/50) and the proliferation decreased with the increasing of the dilution of the conditioned medium. In cells PT67 it has been seen that the sample MVL-hIL-12lig increases the induction in a 41% with regard to the values of the negative control (MVL-hIL10) (FIG. 6).

The presence of hIL12 in the supernatants of the transfected cells has been confirmed by ELISA. The ELISA test results are shown in Table 5. pBabe-Neo and MVL-hIL-10 have been used as negative controls in the test.

TABLE 5

| Test | Supernatant | ng/ml |
|---|---|---|
| 1 | PT67 IL10/IL12 | 0.12 |
| 2 | CHO IL10-IL12 | 1.01 |
| 3 | CHO-IL12 | 2.46 |

The results obtained in the test ELISA demonstrates that cells PT67 as well as CHO cells expresses the human recombinant protein.

The supernatant of CHO cells transfected with MVL-hIL10 has a hIL-10 concentration of about 1 ng/ml.

The viral or non viral vectors, such as vectors MVL-hIL12, MVL-hIL10, MVL-hIL12-hIL10 y MVL-hIL12lig, may be employed in cancer treatment by applying the vectors intra or peritumorally in an individual affected by the tumor or employing retroviral producer cells (RVPC). Viral vectors as retrovirus, adenovirus, adenovirus associated vireus, herpes virus, lentivirus or other ones (e.g. non vial vector) carrying IL-10 and IL-12 coding sequence could be used.

The non viral vectors may be administered by liposomes, by means of the gene gun technique or any other technique allowing incorporating the vector in tumor cells or other host cells, wherein said incorporation allows the expression of coding sequences in their respective products (hIL-10 and hIL-12). The incorporation into the cells may be transitory or may be permanent by insertion in the genome of the host cell.

It is apparent to any person skilled in the art that only one vector may be employed, the vector comprising all the coding sequences, such as the retroviral vector MVL-hIL12-hIL10.

Both, viral vectors vector and non viral vectors may also be employed for the preparation of anti-tumor vaccines for ex-vivo applications or gene delivery therapy, such as autologous vaccines comprising host cells that are transfected/infected with the vector(s) having the hIL-10 and sub-units of hIL-12 coding sequences, such as MVL-hIL12, MVL-hIL10, MVL-hIL12-hIL10 y MVL-hIL12lig.vectors. The host cells may be tumor cells from the patient (tumor cell vaccines). Other cells may also be employed as anti-tumor cell vaccines, such as tumor infiltrating lymphocytes (TIL), LAK cells, endothelium precursor cells, fibroblasts, keratinocites, dendritic cells or any type of cells permitting the expression of hIL-10 and hIL-12 coding sequences.

The host cells (anti-tumor cell vaccine) may be injected into the patient either directly into the tumor or in any region close to the tumor, before or after a traditional radical therapy or another treatment.

According to the results obtained by applying the invention, it may be stated that other genetic constructions and vectors carrying coding sequences, for example, interleukin coding sequences that evoke different immune pathways (Th1+Th2). Viral or non viral vectors can be employed, for example, with vectors carrying IL-4 or IL-5 coding sequences instead of the above mentioned IL-10 coding sequence; and also carrying IL-7 or IL-18 or IFNγ coding sequences in replacement of the above mentioned IL-12 coding sequence for cancer treatment.

The hIL-10 coding sequences and the hIL-12 or hIL-12 fragments could be cloned in viral or non viral vectors in a manner that the obtained product is a chimeric protein liable to be digested by specific proteases within the host cells or once released to the medium in a manner that the interleukins are subsequently released as independent molecules. For example, glycine links containing specific proteases sites such as thrombin target sequence, may be employed.

Any treatment strategy employing vectors expressing the combination of IL-10 and IL-12 or the combination of two or more molecules together evoking pathways Th1 and Th2 of the immune system may be employed for the treatment of neoplasia such as colon, breast and adenocarcinomas in general.

According to the present invention it is convenient that both cytokines (IL-10 and IL-12) expresses alltogether and efficiently. In a preferred embodiment of the invention the vectors carrying IRES elements create polycistronic messages.

The invention may be better understood with reference to the following examples, which are representative of some of the embodiments of the invention, and are not intended to limit the invention.

### EXAMPLE 1

Vector Construction, Transfection of Packaging Cells and Transduction of Tumor Cells for Animal Model.

The construction and characterization of the retroviral system, transfection of packaging cells, screening of IL-10 and IL-12—producing clones, transduction of target cells and assessment of mIL-10 and mIL-12 levels have been previously described by Adris S. Gene Ther 6(10): 1705-1712, 1999 and Adris S. Cancer Res 60(23): 6696-6703, 2000, herein incorporated by reference)). CT26, LM3 and amphotropic GP+env AM12 cells were grown as described (Adris S. Gene Ther 6(10): 1705-1712, 1999, herein incorporated by reference). CT26-IL12 and LM3-IL12 cells produced 0.21 and 0.37 ng/ml per 10⁵ cells per 24 hr of mIL-12, respectively. CT26-IL10 and LM3-IL10 produced 5.8 and 6.3 ng/ml per 10⁵ cells per 24 hr of mIL-10, respectively. All the cell lines were routinely tested for the absence of mycoplasma (MYCOTECT, Life Technologies, Inc.)

### EXAMPLE 2

Animal Model for In Vivo Studies

8- to 10-week old male balb/c mice obtained from the animal facility of the National Institute of Drugs and Clinical Trials (INAME) were s.c. injected in one flank with 5×10⁵ tumor cells in a total volume of 0.1 ml. Mice bearing 0.2-0.3 cm³ CT26 or LM3 tumors were treated once per week during four weeks with proliferating (LV), mytomicin C-treated or irradiated autologous tumor vaccine producing IL-10, IL-12 or combination of both cell types. Vaccines were applied at a distance of 1.5 cm apart from the established tumor or in the opposite flank. In another series of experiments, mice bearing established tumors were vaccinated in the peritumoral area either with GP+env AM12 packaging cells producing the different retroviral vectors carrying the cytokines cDNA or with fibroblasts constitutively producing both cytokines admixed with irradiated autologous tumor cells. Inactivation of cells by mitomycin C was performed as previously described by Adris S. Gene Ther 6(10): 1705-1712, 1999, while irradiated cells received 5000 rads from a ¹³⁷Cs source (CERBIS SA, Buenos Aires). Inactivation of tumor cells did not abrogate secretion of cytokines in vitro over the course of 7 days.

### EXAMPLE 3

Biological Assays

Cell depletion

Mice were selectively depleted by in vivo treatment with specific antibodies for different lymphocytes subpopulations as reported previously by Adris S. Gene Ther 6(10): 1705-1712, 1999, included herein as a reference. The antibodies that were used were monoclonal antibody (mAb) YTS 191.1 for CD4⁺ cells, mAb YTS 169.4 for CD8⁺ cells, and PK136 4D11 hybridoma clones from NK cells and NK cell subsets purchased from ATCC (HB-191 and HB240, respectively).

Assessment of IL-4, IFN-T and IgGs Levels

The isolation of spleen cells and assessment of IL-4 and IFNT was performed essentially as described by Adris S. Gene Ther 6(10): 1705-1712, 1999, herein included as a reference. Total levels of circulating and anti-CT26-specific IgG2a and IgG1 were performed as described previously by Adris S. Gene Ther 6(10): 1705-1712, 1999, herein incorporated by reference.

Leukocyte Chemotaxis Assay

It was essentially performed as described by Jinquan T. J. Immunol. 151: 4545-4551, 1993 and Larsen CG. Science 243: 1464-1466, 1989.

Lymphocytes were isolated from mice peripheral blood using a Ficoll-Hypaque gradient and placed onto the upper chamber of a transwell. As lymphocytes chemoattractants we placed in the lower chamber serum-free conditioned medium obtained either from inactivated or non-inactivated—control, IL-10 and IL-12-producing—CT26, and LM3 cells. Similar studies were performed using mIL-10 as a chemoattractant (R&D, Minneapolis, Minn.) (5-10 ng/mi) instead of cell conditioned media. Anti-IL10 antibody (Pharmigen, Cambridge, UK) was used at a final concentration of 2 μg/ml. Cells were allowed to migrate for 2 hr at 37° C. in a 5% CO₂ humidified incubator and the number of migrated cells was evaluated with a Neubauer chamber.

Histological Studies

Tissues were fixed, embedded in paraffin, sectionated at 5 μm and stained with hematoxilin-eosin. For immunohistochemical analysis, sections were incubated with rat anti-mouse macrophage antibody (F4/80; Serotec, Oxford, United Kingdom; 1/50 final dilution) overnight at 4° C. followed by biotin-labeled goat anti-rat antisera (Jackson Immunoresearch Lab, West Grove, Pa.). After washing, sections were incubated with ABC Vectastain Elite reagent (Vector Laboratories, Burlingame, Calif.). Staining was developed with diaminobenzidine and counterstaining with hematoxilin.

Statistical Analysis

Survival analyses were performed using Kaplan-Meier's method. Statistical comparisons were made using the log-rank test. In the other experiments the significance of differences were determined by using the student's test. Differences were considered significant when p<0.05.

### EXAMPLE 4

Cloning of cDNAs of Human Interleukin 10 and Interleukin 12 (hIL-10 e hIL-12).

Extraction of ARN of Lymphocites from a Sample of Peripheral Blood.

Peripheral blood was extracted and even volumes of heparinized blood and saline solution were mixed. A ficoll solution, in a 3:4 relation, was added to the mixture (Ficoll Paque® (Pharmacia Biotech), blood) and the mixture was centrifuged at 400 g for 40 minutes at 18° C.-20° C. for allowing a gradient formation. The white halo corresponding to the lymphocites (FIG. 1) was separated and a washing with 3 volumes of saline buffer was carried out. Each washing consisted of resuspending and centrifuging at 60-100 g for 10 minutes at a temperature of 18° C.-20° C. Then the cells were counted in a Neubawer chamber and the concentration was adjusted to 10⁶ lymphocites/ml in a medium RPMI 1640 (Life Technologies) supplemented with glutamine (2 mM), penicillin (100 U/ml) and streptomycin (100 μg/ml). PMA (20 mg/ml) and A23187 (25 ng/ml) were added, and the induction was carried out for 17 hours. After this period of time the cells were separated by centrifuging at 1000 rpm for 10 minutes, they were washed with PBS and the ARN was removed by using the TRIZOL® protocol (Life Technologies). The obtained ARN was stored at −80° C. until the use thereof for preparing cDNA.

Preparation of cDNA from ARN of lymphocites.

Between 4 y 6 μl of the preparation of ARN were mixed with 10 μM of oligo-dT (0,5 mg/ml) and it was incubated for 10 minutes at 65° C. The mixture was cooled in ice and 200 U of the reverse transcriptasa (SuperScript™ II, Life Technologies), 20 U of RNAsin (Promega), 0.5 mM of dNTPs and 0.01 M of ditriotreitol (DTT) were added. This preparation was incubated at a room temperature for 10 minutes and then, for 1 hour 30 minutes at 42° C. Finally the enzyme was inactivated for 15 minutes at 65° C. and the obtained cDNA preparation was conserved at −20° C. until the use thereof.

Cloning of cDNA of hIL-10.

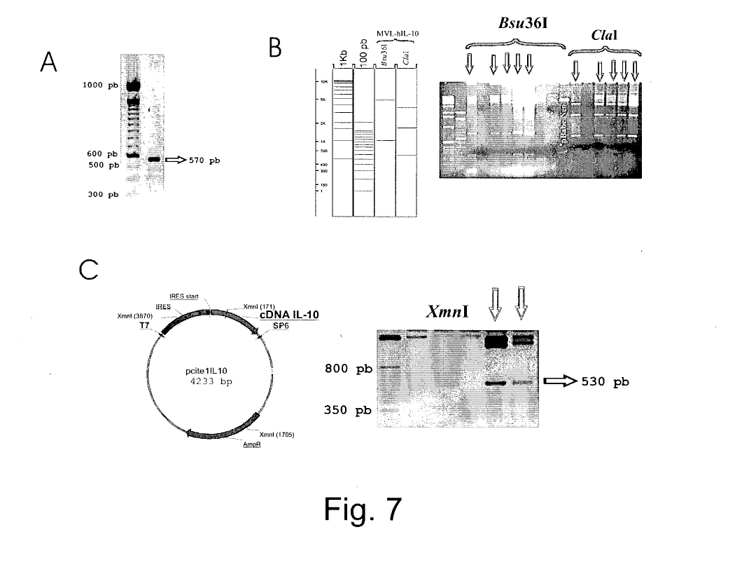

The cDNA of hIL10 obtained in the prior step with the primers IL10F/IL10R (Table 3) and the polymerase Pfu was amplified. A PCR product of 570 pb was obtained, which product was purified from an electrophoretic run in a agarose gel, using the commercial kit QIAquick Gel extraction (QIAGEN®) (FIG. 7A). Afterwards, poliA was added and the product was linked to a commercial vector pGEM®-T Easy Vector (Promega). The IL10 cDNA was obtained from pGEM-IL10 with the NcoI y SalI, and it was linked into the vector MVL, thus obtaining MVL-IL10 (FIG. 7B). On another hand, the IL10 cDNA was linked into the vector pCite-1 for further use in the cloning of vector MVL-IL12-IL10. The resulting vector was named pCite-1-hIL10 (FIG. 7C). The cloned fragment was sequenced in the plasmid pCITE-1-hIL10 by using the primer sp6. The obtained sequence was identical to the hIL-10 sequence as found in data banks.

Cloning of hIL-12 cDNA.

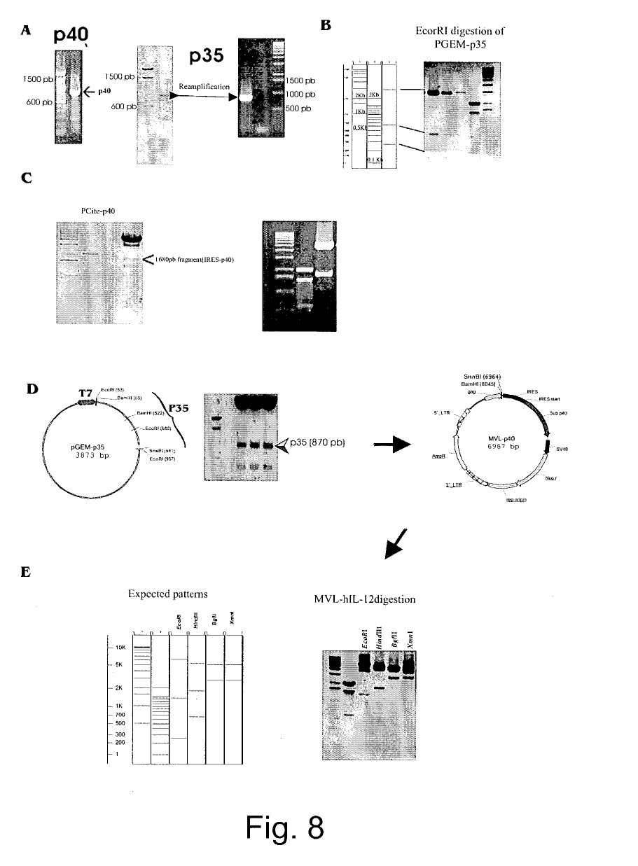

The hIL12 cDNA of the sub-units p35 y p40 were amplified from the preparation cDNA of lymphocites of peripheral blood by using the respective primers shown in Table 3 (primers P40R, P40F, P35R and P35F). FIG. 8A shows the PCR products obtained for the sub-units p35 y p40. In the case of sub-unit p35 the product from the PCR reaction with the oligonucleotides P35f3/P35R resulted complex (FIG. 8A), and this is why the DNA of the 854 pb band was isolated by an electrophoretic run and it was used as a mold for a second amplification (FIG. 8A). This product was cloned in pGEM®-T Easy Vector providing the vector pGEM-p35 (FIG. 8B). The identity of hIL-12 sub-unit p35 was verified by sequencing the primer T7 of the insert cloned in pGEM-p35.

The amplified ADN corresponding to the sub-unit p40 was sub-cloned in vector pGEM®-T Easy Vector and then in pCITE-1, thus obtaining pCite-l-p40. The ADN corresponding to the sequence of IRES-p40 was obtained from the plasmid pCite-1-p40 from a restriction with SalI and a partial restriction with EcoRI (the sub-unit p40 has an inner recognizing site for EcoRI). The resulting fragmentwas cloned in the plasmid MVL, thus obtaining MVL-p40 (FIG. 8C).

The cloning of sub-unit p35 cDNA in vector MVL-p40 was done as shown in FIG. 8D. Briefly, the sub-unit p35 cloned in vector pGEM-p35 was isolated from the digestion of said plasmid with enzyme SnaBI and, by a partial digestion, with enzyme BamHI. The product having 870 pb was purified and linked to the plasmid MVL-p40 linearly with enzymes SnaBI and BamHI. The resulting plasmidic vector was named MVL-hIL12 (FIG. 8E).

Construction of Retroviral Vector pBabe-Neo-IL12-IL10

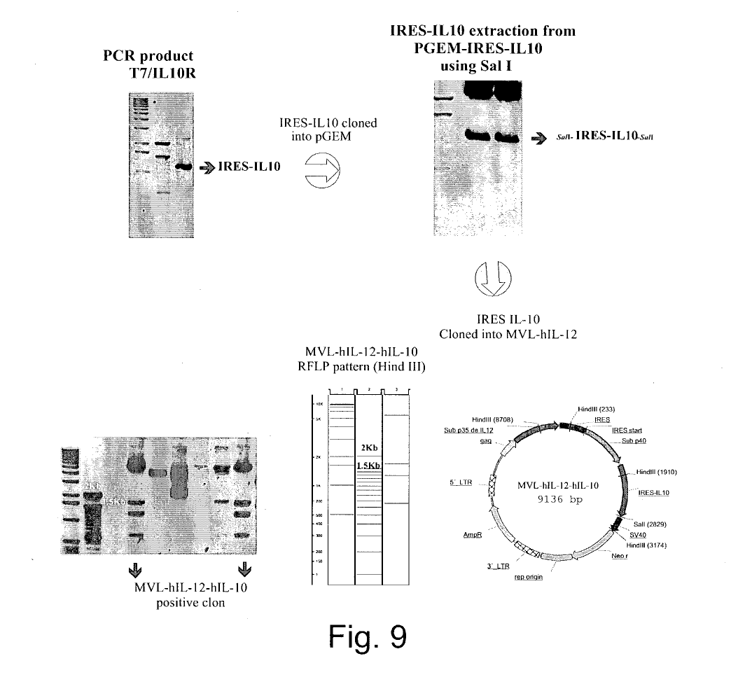

From pCite-1-hIL-10 and by PCR the DNA containing the IRES motive linked to the hIl10 cDNA de hIL10 was obtained by using the primers T7 y IL10R. The latter one has a recognizing site for the enzyme SalI, precisely the site selected for the insertion of IRES-IL10 in MVL-hIL12 (FIG. 9). The PCR product was cloned in the PGEM®-T Easy Vector and a clone was selected (pGEM-IRES-IL10) for permitting the releasing of IRES-IL10 with the use of only enzyme SalI (FIG. 9). The released fragment of about 1200 pb, was then linked to MVL-hIL12 linearized with SalI. The plasmid product of this linking was named MVL-hIL12-hIL10 (FIG. 9).

Cloning of hIL-12lig cDNA

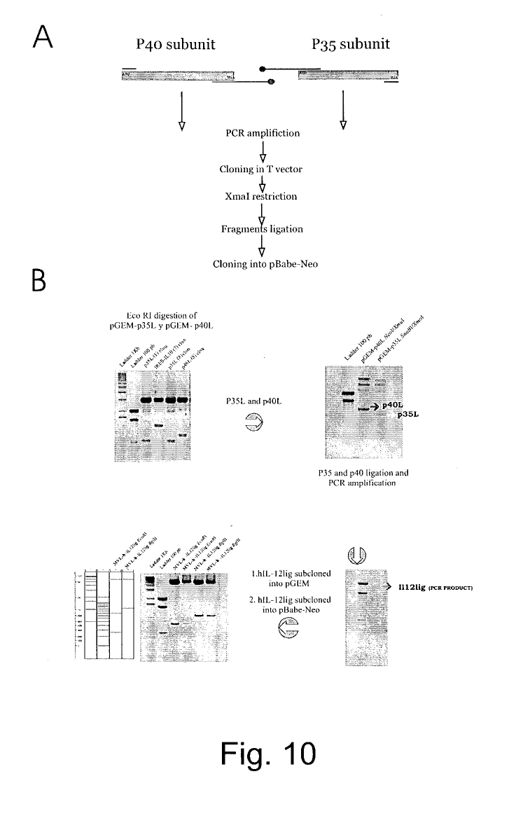

With the purpose of constructing a hIL-12 cDNA containing the sub-units p35 and p40, in a manner that they transcribe alltogether, the strategy shown in FIG. 10 was designed. The DNA fragments of the two sub-units composing the cytokines were joined through a region rich of prolines, valines and lysines, without placing the stop codon of sub-unit 40 and the first initiation region of p35. The reverse primer was designed for amplifying sub-unit p40 with a site that was homologous to the cDNA sequence, a site encoding valines, prolines and lysines and finally a restriction site for XmaI (CCCGGG). The reverse primer of this sub-unit was named p40Rlig. The primer for sub-unit p35 also has a recognizing site for the same enzyme (XmaI), a region that is rich of valine, proline and lysine and the region that is homologous to p35 cDNA. The PCR products of sub-units p35 and p40 were cloned, namely p35L and p40L, in the pGEM®-T Easy Vector, thus obtaining pGEMp35L and PGEMp40L (FIG. 10B). Both inserts were confirmed by profiles RFLP and by sequencing. The p35L and p40L products were obtained from those plasmids, with cohesion ends leading to pGEMp35L with SnaBI y XmaI; and pGEMp40L with NcoI y XmaI. The released fragments were purified and joined to each other. A joint product aliquot was amplified through PCR by using the primer forward of p40 (p40F, Table 3) and the primer reverse of sub-unit p35L (p35R, Table 3). The PCR product was purified and cloned in plasmid pGEM®-T Easy Vector, resulting in pGEM-hIL12lig. The insert hIL12lig was obtained from pGEM-hIL12lig with enzymes SalI y SnaBI. Site SalI was modified with enzyme Klenow and then joined to plasmid pBabe-Neo linearized with enzyme SnaBI. The product resulting from the joining was named MVL-hIL12lig (FIG. 10B).

### EXAMPLE 5

Transfection of Cells CHO and PT67 with Plasmids MVL-hIL-12, MVL-hIL-10, MVL-hIL-12-hIL-10, MVL-hIL-12lig and pBabe-Neo.

Transfection with lipoafectamine 2000 (Invitrogen, Life Technologies) was carried out in 24-well plates following the instructions from the manufacturer.

### EXAMPLE 6

Bioassay for hIL12

Peripheral blood was extracted and even volumes of heparinized blood and saline solution were mixed. A ficoll solution, in a 3:4 relation, was added to the mixture (Ficoll Paque® (Pharmacia Biotech), blood) and the mixture was centrifuged at 1400 g for 40 minutes at 18° C.-20° C. for allowing a gradient formation. The PBMC were separated and a washing with 3 volumes of saline buffer was carried out. 10⁷ PBMC were cultivated in 20 ml of medium supplemented in culturing 75 cm² flask. 40 μl of PHA (5 mg/ml) were added and the cells were incubated in horizontal manner for 4 days.

Afterwards, 20 ml of supplemented medium were added to the culture and this was transferred to another 75 cm² flask. hIL2 (R&D #202-IL) was added up to a final concentration of 50 U/ml and it was incubated for 24 hours. Then the cells were washed and 50 μAl were seeded (4×10⁶ cells/nl) 96-well plates and 50 μl of hIL12 dilutions used as reference standard or 50 μl of the supernatant of the culture to be tested were added.

The cells were incubated for 4-6 hours and thymidine ([methyl-³H] Thymidine, 1 mCi/ml 25 Ci/mmol, Amersham Pharmacia Biotech UK #TRK120), in a 1 μci/well concentration, was added. The cells were incubated for 22-24 hours more.

The cells were harvested with a harvester (Nunc Cell Harvester 8), the cells were collected in a filter paper made of glass microfibers GF/A (Whatman # 1820125). The incorporated radioactivity was measured in β-radiation counter.

Employed supplemental mediums: Three equal parts of the medium complete RPMI and complete DMEM were mixed. 10 mM of HEPES, 0.006% (p/v) of L-arginine, 0.1% (p/v) of dextrose and 5% of human serum AB, or autologous SFB or autologous serum were added.

### EXAMPLE 7

ELISA Test for hIL-10 and hIL-12

Cytokines hIL-10 and hIL-12 were revealed in the culturing mediums by an ELISA test (Peprotech) under the manufacturer instructions.

What is claimed is:

1. A method for treating cancer, comprising administering an effective amount of a combination of a first expression vector comprising a type 2 (Th2) cytokines coding sequence, a biologically active fragment, a variant or a peptide thereof; and a second expression vector comprising a type 1 (TH1) cytokines coding sequence, a biologically active fragment, a variant or a peptide thereof, in a mammalian patient in need of cancer treatment.

2. A method according to claim 1, wherein said type 2 cytokines coding sequence is selected from the group consisting of hIL-10, hIL-4 and hIL-5 coding sequence and said type 1 cytokines coding sequence is selected from the group consisting of hIL-12, hIL-7, hIL-18 and hIFNγ coding sequence.

3. A method according to claim 2, wherein said type 2 cytokines coding sequence is hIL-10 coding sequence, a biologically active fragment, a variant or a peptide thereof and said type 1 cytokines coding sequence is hIL-12 coding sequence, a biologically active fragment, a variant or a peptide thereof.

4. The method according to claim 1, wherein said first expression vector and said second expression vector are intratumorally administered into the patient.

5. The method according to claim 1, wherein said first expression vector and said second expression vector are peritumorally administered into the patient.

6. The method according to claim 1, wherein said first expression vector and said second expression vector are formulated in a pharmaceutically acceptable carrier and/or excipient.

7. The method according to claim 1, wherein said first expression vector and said second expression vector drive the expression of the encoded TH1 and TH2 cytokines, respectively.

8. The method according to claim 7, wherein said first expression vector and said second expression vector drive the expression of the encoded hIL-10 and hIL-12 cytokines, respectively.

9. The method according to claim 1, wherein said expression vectors are selected from the group comprising of retrovirus, adenovirus, adenovirus associated virus, herpes virus, lentivirus, vaccinia virus and non-viral vectors

10. The expression vectors of claim 1, wherein said first expression vector is as shown in FIG. 11A and said second expression vector is as shown in FIG. 11B and D.

11. A composition comprising an effective amount of a first expression vector and a second expression vector of claim 1 in a pharmaceutically acceptable carrier and/or excipient.

12. An anti-tumor cell vaccine comprising cells transfected with the first expression vector and cells transfected with the second expression vector of claim 1, adyuvants and excipients.

13. The anti-tumor cell vaccine according to claim 12, wherein the cells are autologous cells selected from the group consisting of tumor cells, tumor infiltrating lymphocytes, LAK cells, endothelium precursor cells, fibroblasts, keratinocites and dendritic cells.

14. The anti-tumor cell vaccine according to claim 12, wherein the cells delivery an amount of hIL-12, a biologically active fragment, a variant or a peptide thereof and an amount of hIL-10, a biologically active fragment, a variant or a peptide thereof.

15. A method for treating cancer, the method comprising administering an effective amount of the anti-tumor cell vaccine of claim 12, in a mammalian patient in need of cancer treatment.

16. The method according to claim 15, wherein said anti tumor cell vaccine is administered into the patient, close to the tumor.

17. A method for treating cancer, comprising administering an effective amount of an expression vector comprising a first type 2 (Th2) cytokines coding sequence, a biologically active fragment, a variant or a peptide thereof; and a second type 1 (TH1) cytokines coding sequence, a biologically active fragment, a variant or a peptide thereof, in a patient in need of cancer treatment.

18. A method according to claim 17, wherein said first type 2 cytokines coding sequence is selected from the group consisting of hIL-10, hIL-4 and hIL-5 coding sequence and said second type 1 cytokines coding sequence is selected from the group consisting of hIL-12, hIL-7, hIL-18 and hIFNT coding sequence.

19. A method according to claim 18, wherein said first type 2 cytokines coding sequence is hIL-10 coding sequence, a biologically active fragment, a variant or a peptide thereof and said second type 1 cytokines coding sequence is hIL-12 coding sequence, a biologically active fragment, a variant or a peptide thereof.

20. The method according to claim 17, wherein said expression vector is intratumorally administered into the patient.

21. The method according to claim 17, wherein said expression vector is peritumorally administered into the patient.

22. The method according to claim 17, wherein said expression vector is formulated in a pharmaceutically acceptable carrier and/or excipient.

23. The method according to claim 17, wherein said expression vector drives the expression of the encoded TH1 and TH2 cytokines.

24. The method according to claim 23, wherein said expression vector drives the expression of the encoded hIL-10 and hIL-12 cytokines.

25. The expression vector of claim 17, wherein said expression vector is as shown in FIG. 11C.

26. A composition, comprising an effective amount of the expression vector of claim 17 in an acceptable carrier and/or excipients.

27. An anti-tumor cell vaccine comprising cells transfected with the expression vector of claim 17, adyuvants and excipients.

28. The anti-tumor cell vaccine according to claim 27, wherein the cells are autologous cells selected from the group consisting of tumor cells, tumor infiltrating lymphocytes, LAK cells, endothelium precursor cells, fibroblasts, keratinocites and dendritic cells.

29. The anti-tumor cell vaccine according to claim 27, wherein the cells delivery an amount of hIL-12, a biologically active fragment, a variant or a peptide thereof and an amount of hIL-10, a biologically active fragment, a variant or a peptide thereof.

30. A method for treating cancer, the method comprising administering an effective amount of the anti-tumor cell vaccine of claim 27, in a mammalian patient in need of cancer treatment.

31. The method according to claim 30, wherein said anti tumor cell vaccine is administered into the patient, close to the tumor.
